(19) United States
(12) Patent Application Publication
(10) Pub. No.: US 2025/0052712 A1
(43) Pub. Date: Feb. 13, 2025

(54) SENSOR ASSEMBLY AND METHOD OF MANUFACTURE

(71) Applicants: Analog Devices International Unlimited Company, Limerick (IE); Arma Biosciences, Toronto (CA)

(72) Inventors: David BOLOGNIA, Charlestown, MA (US); Joyce WU, Somerville, MA (US); Helen BERNEY, Pennywell (IE); Surath GOMIS, Toronto (CA); Shana KELLEY, Glencoe, IL (US)

(21) Appl. No.: 18/717,862

(22) PCT Filed: Dec. 7, 2022
(86) PCT No.: PCT/EP2022/084871

§ 371 Date: Jun. 7, 2024

(51) Int. Cl. G01N 27/327; G01N 33/543

(52) U.S. Cl. CPC G01N 27/3272; G01N 27/3276; G01N 33/5438

(57) ABSTRACT

The present disclosure provides a sensor assembly for measuring a property of a sample. The sensing assembly comprises first and second dielectric layers. The first dielectric layer provides a well or aperture which is associated with an electrode. The second dielectric layer is provided on the first dielectric layer and provides an aperture fluidly connected to the well or aperture in the first dielectric layer.

### CROSS-REFERENCE TO RELATED APPLICATIONS

This application is a 35 U.S.C. § 371 National Stage Application of International Patent Application No. PCT/EP2022/084871, filed Dec. 7, 2022, which claims the benefit of and priority to U.S. Provisional Patent Application No. 63/287,020, filed Dec. 7, 2021, the contents of which applications are hereby incorporated by reference herein in their entireties.

### FIELD OF THE DISCLOSURE

This disclosure relates to a sensor assembly, for example a biosensor or chemical assay, for measuring a property of a sample (e.g., for sensing an analyte), and a method of manufacturing a sensor assembly.

### BACKGROUND

Conventional electrochemical sensor assemblies are often produced by providing separate sensor structures, i.e., the electrical interfaces and functionality, and packages, such as the fluidic structures. This requires separate manufacturing processes and increases cost and complexity.

Moreover, particularly where the sample size is small (for example, on the microliter scale), there is a desire to condense the sensor structure and package sizes but retain a high number of electrodes.

It would be advantageous to provide a sensor assembly which avoids these drawbacks and method of manufacturing such an assembly.

### SUMMARY OF THE DISCLOSURE

The present disclosure provides a sensor assembly for measuring a property of a sample. The sensing assembly comprises first and second dielectric layers. The first dielectric layer provides a well or aperture which is associated with an electrode. The second dielectric layer is provided on the first dielectric layer and provides an aperture fluidly connected to the well or aperture in the first dielectric layer.

In one embodiment, a sensor assembly for measuring a property of a sample comprises a substrate, at least one electrode provided on the substrate, a first dielectric layer provided on the substrate, the first dielectric layer comprising a well or aperture associated with the electrode such that the electrode is responsive to the presence of a sample received within the well or aperture, and a second dielectric layer provided on the first dielectric layer comprising an aperture extending through the second dielectric layer fluidly connected to the well or aperture in the first dielectric layer.

In another embodiment, a method of forming a sensor assembly comprises providing a substrate; forming at least one electrode on the substrate; providing a first dielectric layer on the substrate; and providing a second dielectric layer on the first dielectric layer. The first dielectric layer comprises a well or aperture associated with the electrode such that the electrode is responsive to the presence of a sample received within the well or aperture; and the second dielectric layer comprises an aperture extending through the second dielectric layer and is fluidly connected to the well or aperture in the first dielectric layer.

### BRIEF DESCRIPTION OF THE DRAWINGS

The present invention will now be described in more detail with reference to the accompanying drawings, which are not intended to be limiting:

FIG. 1A provides a schematic cross-sectional view of a sensor assembly according to an embodiment;

FIG. 1B provides a schematic cross-sectional view of a sensor assembly according to an embodiment;

FIG. 2 provides a schematic cross-sectional view of a further sensor assembly according to an embodiment;

FIG. 3 provides a schematic cross-sectional view of a further sensor assembly according to an embodiment;

FIG. 4 provides a schematic plan view of a further sensor assembly according to an embodiment; and

FIGS. 5A to 5E schematically depict the manufacture of a sensor assembly according to an embodiment.

### DETAILED DESCRIPTION

Various sensor assemblies are known. Conventional sensor assemblies, and particularly electrochemical sensors, are often produced by providing separate sensor structures, i.e. the electrical interfaces and functionalization, and packages, such as the fluidic structures. This requires separate manufacturing processes and can be costly to manufacture.

Moreover, particularly where the sample size is small (for example, on the microliter or smaller scale), there is a desire to condense the sensor structure and package sizes but retain a high number of electrodes. It is expensive to manufacture this type of arrangement using conventional semiconductor processing techniques. For example, forming fluidics in silicon substrates is prohibitively costly, at least in part due to the scale of the fluidic structures, and suffers from other drawbacks such as difficulties electrically isolating individual electrodes when a high density of electrodes is required.

In one embodiment, a sensor assembly for measuring a property of a sample comprises a substrate, at least one electrode provided on the substrate, a first dielectric layer provided on the substrate, the first dielectric layer comprising a well or aperture associated with the electrode such that the electrode is responsive to the presence of a sample received within the well or aperture, and a second dielectric layer provided on the first dielectric layer comprising an aperture extending through the second dielectric layer fluidly connected to the well or aperture in the first dielectric layer.

Embodiments provide a sensor assembly which combines the electrodes and fluidics in a single integrated package. Such a structure can be easier to manufacture than traditional sensor assemblies, particularly as compared to those whereby the sensor elements (e.g., the electrode(s) and any traces) are manufactured separately or on a separate part of the packaging components (e.g., the fluidics). For example, in embodiments, the first dielectric layer forms a lower layer which is built up on top of the electrode(s) and forms a lower aperture or lower well which provides a first part of the fluidic structures used to bring a sample to the electrode(s) or retain a sample thereon. The second dielectric layer is an upper layer provided on the first dielectric layer and provides an upper aperture which also forms a part the fluidic structures used to bring a sample to the electrode(s) or retain a sample thereon.

In such embodiments, the first dielectric layer with the first (or lower) well (i.e., channel or recess, which may have a base in the first dielectric layer) or aperture (i.e., via or through hole which may expose an adjacent electrode surface) acts as the sensor surface and/or the interface with the respective electrode. The electrode in communication will the first aperture or first well can therefore produce a signal indicative of a property of a sample or analyte within the first aperture or first well. In some embodiments, the electrode or the first dielectric layer is functionalised so as to interact with a sample received within the first well or aperture of the first dielectric layer. In some embodiments, the electrode comprises a capture species configured to selectively interact with an analyte within a sample. This may be a functional layer provided on the electrode, for example.

The second dielectric layer has at least one second or upper aperture located therein which extends through the second dielectric layer (e.g., from an outer surface to an inner surface, which inner surface is in contact with (directly or indirectly) the first dielectric layer) to join with the first (or lower) well or aperture in the first dielectric layer. Thus, the second or upper aperture fluidly communicates with the first well or aperture in the first dielectric layer so that fluid can be provided from the outer surface of the second dielectric layer to the first well or aperture of the first dielectric layer. The second dielectric layer thus incorporates the fluidics (e.g., fluid channels) required to provide sample to the electrode and can be used as a means of retaining the sample within the first well or aperture of the first dielectric.

Such a structure (and a corresponding method of manufacture) also allows for improved customisability tailored to the end use. For example, the first structure (i.e., first well or aperture) in the first dielectric layer serves as the interface with the electrode and so in part determines the response that will occur when the sample is received therein. This can be adjusted depending on the needs, for example by using different thicknesses, well/aperture size, etc., This is especially straightforward in view of the materials and process used. However, the fluidics need not change and the second dielectric layer can remain the same. Alternatively or additionally, the second dielectric layer can be modified to change the fluidics (e.g. the fluid retention properties, fluid pathways etc.). Usefully, the structures formed in the second dielectric layer can have a much larger scale and complexity than the fluid structure (e.g. first well or aperture) in the first dielectric layer, thereby reducing the complexity of the formation of the first well or aperture in the first dielectric layer. It will be appreciated that the first dielectric layer will often require a more precise formation and will be more expensive to form and so the separation of the complex and sensitive first dielectric layer and the macro fluid control properties improves manufacturability. This is particularly advantageous for small-scale sensor assemblies. The alignment of fluidics and different components on the micro-scale (or smaller) is particularly complex but embodiments provide for an arrangement which provides smaller tolerances. For example, manufacturing micro-scale fluidics using some techniques can make it to align apertures (for example, when aligning a separate fluidics module with a sensor assembly), whereas the formation of the fluidics using two separate dielectric layers bridges the gap and enables much more precise alignment.

The presence of the first well or aperture in the first dielectric layer and the second aperture in the second dielectric layer in embodiment therefore provides a significant range of customisability without requiring fundamental changes to the sensor assembly design. For example, the first well or aperture in the first dielectric can be used as the electrode interface and thus only need receive a small amount of fluid (e.g., liquid) sample in order to conduct the measurement. Thus, it may have only a small well or aperture size. The aperture in the second dielectric can be larger and can be used to retain the sample in the first well or aperture (or in plural wells or apertures). Thus, the first dielectric can be formed of the particular materials required to provide the functional electrode surface or the interface with the corresponding electrode (as well as the separation between electrodes, where required) and only need have a smaller well or aperture. The second dielectric layer can be provided on a larger scale, with a larger aperture designed to retain the sample on the sensor. This also means different techniques can be used to provide the layers, which further improves customisability and manufacturability.

### Dielectric Layers

The first and second dielectric layers comprise or are formed of at least one dielectric material. In embodiments, these can comprise or be formed of a polymer, a glass, a glass-ceramic, a ceramic, a metal oxide, a metal nitride, a silicon-based material, or combinations thereof.

In some embodiments, the first dielectric layer and/or the second dielectric layer comprise or are formed of a polymer. For example, these may each be polymeric dielectric layers. By polymeric dielectric layers it is meant that the layers are primarily formed of a polymer (e.g., the majority, such as at least 50 wt. %, such as at least 90 wt. % is a polymeric material) but may contain further components, such as dopants or a capture species. In an embodiment, the first dielectric layer and/or the second dielectric layer comprise polyimide, polyethylene terephthalate. polyvinylchloride (PVC) or a combination thereof.

In some embodiments, the first dielectric layer and/or the second dielectric layer comprise or are formed of or from a photo-imageable material. Use of such materials enables the first wells or apertures and the second apertures to be formed using photoimaging or photo etching, which enables more accurate formation of the wells/apertures and therefore more accurate fluidic structures. For example, these can enable alignment of the first well/aperture with the second aperture which could not otherwise be achieved through traditional punch and place stackers. Example photo-imageable materials can be selected from polyimide, polyethylene terephthalate. polyvinylchloride (PVC) or a combination thereof. These can be provided as liquid photo-imageable solder mask (LPSM or LPI) inks and dry-film photo-imageable solder mask (DFSM) onto the substrate or other layers, for example.

In some embodiments, the first and second dielectric layers are formed of or comprise different materials.

In some embodiments, the first dielectric layer has a thickness of from 1 μm to 50 μm. In some embodiments, the second dielectric layer has a thickness of from 50 μm to 1000 μm. In some embodiments, the first dielectric layer has a thickness of from 1 μm to 50 μm and the second dielectric layer has a thickness of from 50 μm to 1000 μm. In embodiments, the first dielectric layer has a thickness of from 1 μm to 50 μm, for example, 15 to 25 μm. In embodiments, the second dielectric layer has a thickness of from 50 μm to 1000 μm, for example from 100 μm to 1000 μm, for example, 100 μm to 500 μm.

In the abovementioned embodiments, the layers (e.g., the first and second dielectric layers) may be provided directly on an adjacent layer (or the substrate) or there may be intervening layers such as a further layer, such as an adhesive. For example, the first dielectric layer may be provided directly on the substrate e.g., adjacent the electrode or may be provided directly on the electrode, or a combination thereof. The second dielectric layer may be provided directly on the first dielectric layer. Layers may be continuous (e.g., across the surface of the substrate, other layers and/or electrode(s)) or may be discontinuous and formed as separate regions on the surface of the substrate and/or electrode(s).

The abovementioned sensor assemblies may be used in a number of different fields for sensing applications. In some embodiments, these are used with small liquid sample sizes, such as less than 5 μL samples (e.g., less than 3 μL, or less than 1 μL). Embodiments are particularly advantageous as the first dielectric layer provides the sensor interface and the second dielectric layer can provide the fluidic structures designed to hold or retain small volume liquids on the sensor interface (i.e. the electrode and/or the sensor region (well or aperture) of the first dielectric). For example, the aperture of second dielectric layer may be adapted to retain a droplet or liquid sample within the first well or aperture. Where there are plural apertures in the second dielectric and plural wells or apertures in the first dielectric layer, each aperture in the second dielectric layer may be adapted to retain a droplet or liquid sample within a respect first well or aperture. In other embodiments, an aperture in the second dielectric layer may retain a droplet or fluid sample in plural first apertures or wells in the first dielectric layer. The dielectric layers set out herein advantageously enable the formation of fluidic structures for volumes at these small sample sizes, for example through enabling micro-fluidic structures to be formed accurately.

In further embodiments, the sensor assembly comprises at least one further layer provided on (e.g., stacked on) the first and second dielectric layers defining at least one further fluidic structure in fluid communication with the second aperture(s) of the second dielectric layer. This can provide further fluid paths or retention structures. The at least one further layer may be a further dielectric layer (such as a third dielectric layer) and may comprise at least one fluidic structure, such as a channel, well, or aperture in fluid communication with the second aperture(s) of the second dielectric layer.

In one embodiment, the first and/or second dielectric layer may be comprised of plural sub-layers. The first or second dielectric layer may accordingly be a composite layer comprised of a number of sub-layers of e.g. different materials. This can be particularly useful for customising the function of the dielectric layer(s) and the cost-effectiveness of the sensor. One exemplary embodiment is where the second dielectric comprises a polyimide and PVC composite—i.e., at least one layer comprising polyimide and at least one layer comprising PVC. In other embodiments, the first and/or second dielectric layer may (each) be a single dielectric layer. For example, the second dielectric layer may be a single layer of polyimide. Such a structure can provide a relatively straightforward and cheap way of producing a sensor structure, which may be flexible and photo-imageable, and may lend itself to single-use or short-lifetime sensors.

In embodiments where the sensor assembly comprises a plurality of electrodes, the first dielectric layer may also separate the plurality of electrodes, thereby electrically separating the plural electrodes. That is, the plural electrodes may be spaced apart (e.g., on the substrate) with at least a portion of the first dielectric layer provided therebetween. This provides a straightforward way of manufacturing and implementing a high density of electrodes. For example, particular applications where such a configuration is advantageous include in sensors for properties of blood whereby only small samples (1-3 μl) may be taken. In some embodiments, the first dielectric layer may also extend over the plural electrodes, such that the electrodes are embedded within the first dielectric layer.

### Functionalisation

As set out above, the sensor assembly may be for obtaining a response relating to a property of a sample. In some embodiments, this may be a detection of an analyte, such as determining analyte concentration, in a sample. The analyte may, for example, be selected from a molecular species, proteins, a metal ion, a virus, and a microorganism. The analyte may, for instance, be a hormone selected from an eicosanoid, a steroid, an amino acid, amine, peptide or protein. The electrode(s) of the sensor assembly may be used to obtain a measurement signal indicative of the interaction between the sample (e.g., an analyte) and the electrode and/or aperture/well of the first dielectric layer in association with the electrode.

A capture species which selectively binds with an analyte may be used to functionalize at least one electrode and/or the aperture/well of the first dielectric layer in association with the at least one electrode, such that the electrode associated with the capture species will provide a measurement signal indicative of the interaction between an analyte and the capture species. Any suitable capture species can be selected for this purpose, according to the analyte which is intended to be sensed by the sensing assembly. For example, the capture species may comprise an antibody with specificity for a particular antigen. In such an example, the analyte may take the form of the antigen. More generally, the capture species may, in some embodiments, comprise at least one selected from a protein, a peptide, a carbohydrate, and a nucleic acid. The protein may, for example, be an enzyme, such as an enzyme having specificity for the analyte. In other non-limiting examples, the protein is an antibody. In an embodiment, the capture species comprises an aptamer. An aptamer may be defined as an oligonucleotide or peptide configured to bind the analyte. Such an aptamer may, for example, be configured to interact with, for example bind, various analyte types, such as small molecules, for example amino acids or amines, proteins, metal ions, and microorganisms. In some non-limiting examples, the aptamer is functionalized with an electro-active moiety, for example a redox-active moiety, and is configured such that a conformational change of the aptamer upon selectively interacting with, for example binding, the analyte causes a change in the proximity of the electro-active moiety with respect to the surface of the respective electrode. By functionalized, it is meant that the capture species may be bound to the electrode and/or a surface of the aperture/well of the first dielectric layer (or plural of the aperture/wells), such as covalently bound.

In some embodiments, the first dielectric layer comprises or is provided with a capture species configured to selectively interact with an analyte within a sample. In one embodiment, the first dielectric layer comprises a capture species provided adjacent a surface of the electrode. For example, this may be provided on or in the well or aperture (e.g., on the surface of or in the material defining the well or aperture). In such an example, the capture species are configured to selectively interact with an analyte of interest in the sample. This can be particularly advantageous where a well is used. In some embodiments, the nadir of the well can be functionalised and the electrode can be arranged so as to be responsive to the presence of a sample (or an analyte therein) in the well.

In some embodiments, the sensor assembly comprises at least a first functionalised region comprising at least one of the wells or apertures in the first dielectric layer, at least one electrode and a first capture species configured to selectively bind to a first analyte and provided on at least a part of the first functionalised region; and a second functionalised region comprising at least one of the wells or apertures in the first dielectric layer, at least one electrode and a second capture species configured to selectively bind to a second analyte and provided on at least a part of the second functionalised region. This means that the functionalisation of the first and second regions is different. In other words, the sensor assembly comprises plural sensing regions each with a different functionalisation and each comprises at least one associated electrode and first well/aperture. This means that there can be plural different sensor types on the same substrate. By functionalisation, it is meant that at least the electrode or first well/aperture of the first dielectric layer is provided with a functional component which can interact with an analyte. An example is a capture species provided adjacent to or on the electrode or the first aperture/well, which capture species can selectively bind to an analyte. The electrode associated with the capture species will provide a measurement signal indicative of the interaction between an analyte and the capture species. Accordingly, the different functionalisation may be achieved by e.g. using different functional groups (e.g. different functionalisation such as different capture species, which capture species selectively bind to different analytes). This can be implemented for example using a discontinuous first dielectric layer with different regions corresponding to the different functionalised region. Thus, in some embodiments, multiple electrodes are uniquely functionalized, and in other embodiments they are commonly functionalized. Multiple layers of dielectrics can also be stacked to create common and non-common wells or apertures. The sensor assembly layered structure disclosed herein can advantageously provide this structure by using the customisability of the first and second dielectric layers. For example, one aperture in the second dielectric layer may be fluidly connected to both of the first and second functionalised regions so that a sample provided to the aperture of the second dielectric layer will be provided to these regions simultaneously. Moreover, the layered structure advantageously reduces the complexity of manufacture, since the more complex and functionalised regions can be formed using the first dielectric layer, before larger-scale fluidics can be formed in the second dielectric layer. This has particular applicability to analysis of multi-analyte fluids, such as bodily fluids, including blood or saliva. A small volume of such fluids can be provided to the second dielectric layer, and this can be distributed by the aperture(s) of the second dielectric layer to the factualised regions for multi-analyte analysis.

### Apertures/Wells

The first dielectric layer comprises at least one aperture or well. In embodiments, there may be plural apertures, wells, or a combination thereof, each of which may be associated with a corresponding electrode. Plural wells or apertures may be associated with the same electrode and plural electrodes may be associated with the same well or aperture, although in some embodiments each well or aperture is associated with only one electrode. By associated with, it is meant that the aperture or well is in fluid communication with the respective electrode(s) or is adjacent such that the electrode is responsive to the presence of a fluid in the aperture or well. A well may take the form of a channel or recess in the first dielectric layer, the base of which may be provided over the associated electrode) or aperture (i.e. via or through hole which may expose an adjacent electrode surface). An aperture may therefore comprise an opening in fluid communication with (e.g., provided on) an electrode and a well may include a wall which provided on or over an electrode (such as a base or nadir). Such a wall may have a thickness of less than or equal to 50 μm, such as less than or equal to 25 μm, less than or equal to 10 μm, or less than or equal to 5 μm so that the associated electrode is responsive to fluid (e.g., sample or analyte) provided therein.

In some embodiments, the volume of the aperture or each aperture (where there are plural apertures) of the second dielectric layer is greater than the volume of the well or aperture or the corresponding well or aperture of the first dielectric layer (i.e., the well(s) or aperture(s) in the first dielectric layer to which the second aperture is fluidly connected). That is, each aperture in the second layer has a volume greater than each of the well(s)/aperture(s) in the first dielectric layer to which it is fluidly connected. This may be at least 1.5 times the volume, at least 2 times, at least 5 times or at least 10 times. The volume of the well or aperture in the first dielectric layer may be from 1 picolitre (μl) to 1 microliter (μl), for example 1 μl to 500 nanolitres (nl), for example 100 μl to 500 nl. The volume of the aperture in the second dielectric layer may be from 250 nl to 5 ml, for example 250 nl to 2000 μl. In some embodiments, each aperture in the second layer has a volume greater than each of the well(s)/aperture(s) in the first dielectric layer to which it is fluidly connected combined.

In embodiments, the second aperture (i.e., the aperture of the second dielectric layer) has a largest diameter of from 50 μm to 20 mm, such as from 50 μm to 10 mm, 50 μm to 5 mm, 50 μm to 500 μm, from 100 μm to 250 μm, from 100 μm to 20 mm, from 500 μm to 10 mm, from 1000 μm to 5 mm. Largest diameter is the diameter at the widest point of the aperture. This may be measured at one or both of the openings of the aperture. In some embodiments, the first aperture or well (i.e. the aperture/well of the first dielectric layer) has a largest diameter of less than or equal to 100 μm, such as less than or equal to 50 μm, 25 μm, 10 μm or 5 μm. In some embodiments, the first aperture or well (i.e. the aperture/well of the first dielectric layer) has a largest diameter of from 0.1 μm to 100 μm, such as 0.1 μm to 50 μm, 0.1 μm to 25 μm, 0.1 μm to 10 μm or 0.1 μm to 5 μm.

In some embodiments comprising plural first apertures/wells and second apertures, the aperture(s) of the second dielectric layer and the aperture(s)/well(s) of the first electrode layer may be arranged so as to be substantially radially symmetric (e.g., radially symmetric) about a central axis. In other words, the aperture(s) (e.g., the arrangement/shape of the aperture(s)) in the second dielectric layer may be radially symmetrical. The aperture(s)/well(s) (e.g., the arrangement/shape thereof) in the first dielectric layer may be radially symmetrical. That is, there may be plural well(s)/aperture(s) and these may be arranged in a radially symmetrical pattern or configuration. This allows a sample to be provided to the central axis and spread equally to each of the first wells/apertures. For example, in the case of a drop of bodily fluid (e.g., blood) being provided. This can also allow the sensor assembly to detect that a liquid sample is fully coating the entire surface of the sensor assembly. In some embodiments, there may be electrodes adapted to sense the presence of a sample (e.g. blood) located at the outermost radial position so as to ensure that the sample covers the descried region.

### Substrate

The substrate may comprise or be formed of any suitable material, such as a polymer, a glass, a glass-ceramic, a ceramic, a metal oxide, a metal nitride, a silicon-based material, or combinations thereof.

In some embodiments, the substrate may comprise or be formed from (e.g., consist of) a polymer such as polyimide (PI) or polyethylene terephthalate (PET). In other embodiments, the substrate may comprise or be formed from (e.g., consist of) a glass, glass-ceramic or ceramic substrate. These example substrates may be flexible substrates.

In some embodiments, the substrate is a flexible substrate. By flexible, it is meant that the substrate is able to deform out of a single plane under a load and will return to the plane when the load is removed. Examples include polyimide (PI) or polyethylene terephthalate (PET) substrates.

### Electrode(s)

In embodiments, the at least one electrode comprises or is formed from copper, nickel, platinum, silver, silver chloride, gold or other noble metals. In some embodiments, the electrode comprises a plurality of metal layers, the metal layers comprising or being formed from copper, nickel, platinum, silver, silver chloride, gold or other noble metals. The electrode layers may be formed on the substrate.

In one embodiment, the sensor assembly comprises a set of electrodes comprising a plurality of electrodes, and wherein the first dielectric layer comprises a plurality of wells and/or apertures, with at least one well or aperture associated with each electrode of the set of electrodes. This is so that each electrode is responsive to the presence of a sample received within the corresponding well or aperture. Each electrode may be associated with a first well or aperture in the first dielectric layer. For example, in some embodiments, there may be at least 4 electrodes in the sensor assembly, for example at least 8 electrodes, at least 16 electrodes or at least 24 electrodes. There may be a mixture of wells and apertures in the first dielectric layer, depending on the required sensor function. In some embodiments, the second dielectric layer comprises a plurality of apertures extending the through the second dielectric layer, each aperture corresponding to and being fluidly connected to one of the plurality of wells or apertures in the first dielectric layer. Thus, each electrode may be associated with a first well or aperture in the first dielectric layer and a second aperture in the second dielectric layer.

In embodiments comprising plural electrodes, this may comprise at least one working electrode (e.g., functionalised electrode) and at least one control electrode (e.g., non-functionalised electrode), and optionally at least one reference electrode. In some embodiments, these may comprise plural working electrodes (e.g., functionalised electrodes) and plural control electrodes (e.g., non-functionalised electrodes). At least one reference electrode may also be provided.

In some embodiments, the plurality of electrodes of the set of electrodes are separated from one another by the first dielectric layer and/or the second dielectric layer.

### Further Components

In some embodiments, the first well or aperture of the first dielectric layer and/or the second well of the second dielectric layer is covered by a liquid-permeable cover or cap. In some embodiments, this is a porous cover or cap, such as a cover or cap comprising at least one opening therethrough such that fluid can pass from the upper surface of the sensor assembly into the well or aperture of the first dielectric layer. In this way, the electrode associated with the wall or aperture can be protected from accidental damage by the cap, but fluid can still pass into the aperture(s)/well(s). This cap may be provided on top of the second dielectric layer, may be co-planar with the second dielectric layer or may be a part of the second dielectric layer. In some embodiments, apertures through the cap or cover may be configured to facilitate capillary action of a fluid to the sensing surface (e.g., the aperture or well in the first dielectric layer. Alternatively or additionally, the aperture of the second dielectric layer may be configured to prevent contact with the well or aperture (e.g., electrode). For example, the height of the dielectric layers may be so as to reduce the risk of contact of a finger with the electrode and/or functionalized surface.

In some embodiments, the sensor assembly comprises a shielding or grounding component or layer. This may be formed by a metallic layer, either as a separate metallic layer to the electrode(s) or may be formed from the same metallic layer as the electrode(s). For example, the shielding or grounding component may be an electrically isolated metallic layer or region. This may be, in some embodiments, formed in the same layer as the electrode(s) but electrically isolated from the electrodes. In some such embodiments, the electrical isolation may be achieved by the first dielectric layer (e.g. by disposing the first dielectric layer between the electrode(s) and shielding or grounding region(s)). In other embodiments, the shielding or grounding component or layer may be a separate metallic layer to the layer in which the electrode(s) are formed. These embodiments can be used to obtain a better signal from the sensor. The metallic or metal layers may comprise or be formed from copper, nickel, platinum, silver, silver chloride, gold or other noble metals.

### Systems

The sensor assembly set out herein can be used as part of a system for measurement of an analyte property. In one embodiment, a system may comprise the sensor assembly as well as a signal processing unit. The signal processing unit may be configured to receive a signal from the electrode(s) providing an indication of (or a property indicative of) the property of the sample thereon (e.g., where the electrode acts a working electrode or control electrode). This may be the potential of the electrode. The system may further comprise a property determination unit for determining the property that is being measured based on the processed signals. These may take the form of one processor, for example, or may be comprised of several processors. A processor may be implemented in any suitable manner, with software and/or hardware, to perform the various functions required. One or all of the units may, for example, employ one or more microprocessors programmed using software (for example, microcode) to perform the required functions. Examples of processor components that may be employed in various embodiments of the present disclosure include, but are not limited to, conventional microprocessors, application specific integrated circuits (ASICs), and field-programmable gate arrays (FPGAS).

In various implementations, the signal processing unit, property determination unit and/or processor may be associated with one or more non-transitory storage media such as volatile and non-volatile computer memory such as RAM, PROM, EPROM, and EEPROM. The non-transitory storage media may be encoded with one or more programs that, when executed on one or more processors and/or controllers, perform the required functions. Various storage media may be fixed within a processor or controller or may be transportable, such that the one or more programs stored thereon can be loaded into the signal processing unit, property determination unit and/or processor.

In some non-limiting examples, the system includes a user interface, such as a display, for communicating the analyte property determined by the property determination unit. Alternatively or additionally, the system may include a communications interface device, such as a wireless transmitter, configured to transmit the analyte concentration determined by the property determination unit to an external device, such as a personal computer, tablet, smartphone, remote server, etc.

### Methods

In one aspect, there is provided a method of forming a sensor assembly, the method comprising: providing a substrate; forming at least one electrode on the substrate; providing a first dielectric layer on the substrate; and providing a second dielectric layer on the first dielectric layer, wherein the first dielectric layer comprises a well or aperture associated with the electrode such that the electrode is responsive to the presence of a sample received within the well or aperture; and wherein the second dielectric layer comprises an aperture extending through the second dielectric layer and is fluidly connected to the well or aperture in the first dielectric layer.

Providing the first dielectric layer and/or the second dielectric layer may comprise applying a film (e.g., a solid or liquid film) to the substrate (optionally over the electrode) to form a first layer. Where a solid film is used, the film may be provided with the aperture(s) or well(s) formed therein. Where a solid or liquid film is used to create the layer, aperture(s) or well(s) formed therein may be provided once the layer is formed on the substrates.

In some embodiments, the first and/or second dielectric layers may be provided by a coating method such as spin coating or screen printing. In other embodiments, these may be applied by applying a pre-formed layer onto the substrate, for example using reel-to-reel coating.

In some embodiments, the method further comprises forming a well or aperture in the first dielectric layer, the well or aperture being associated with the electrode such that the electrode is responsive to the presence of a sample received within the well or aperture. In some embodiments, forming a well or aperture in the first dielectric layer is carried out before providing the second dielectric layer on the first dielectric layer. This may be provided, for example, after providing the first dielectric layer on the substrate. For example, using an etching process or drilling process (such as laser drilling) on the layer formed on the substrate. This may also be provided, for example, using photo-imaging, where a solder mask is applied and selectively removed. Alternatively, the well or apertures may be pre-formed in a film or layer, and the film or layer may be applied to the substrate to form the first dielectric layer. For example, the layer may be pre-punched.

In some embodiments, the method further comprises forming an aperture in the second dielectric layer, the aperture extending through the second dielectric layer to fluidly connect with the well or aperture in the first dielectric layer. This may be provided, for example, after providing the second dielectric layer on the first dielectric layer. For example, using an etching process or drilling process (such as laser drilling) on the layer formed on the substrate. This may be, for example, using a solder mask or photoetching. This can advantageously avoid the difficulties associated with aligning pre-formed aperture. Alternatively, the well or apertures may be pre-formed in a film or layer, and the film or layer may be applied to the first dielectric layer to form the second dielectric layer. In some embodiments, forming the aperture in the second dielectric layer is carried out after providing the second dielectric layer on the first dielectric layer.

In some embodiments, forming the well(s) or aperture(s) of the first dielectric material and the aperture of the second dielectric material may be carried out separately. In this way, the size of the feature (e.g., well or aperture) in each layer can be controlled more easily.

In one embodiment, the method comprises providing a first dielectric layer on the substrate; providing a second dielectric layer on the first dielectric layer, and subsequently forming the at least one aperture in the second dielectric layer. Carrying out the method in this order, i.e., with the formation of the second aperture of the second dielectric layer, has been found to increase the accuracy of the alignment of the second aperture(s) with the respective first aperture(s)/well(s) in the first dielectric layer. For example, the inventors have identified that alignment error can be reduced to well below 25 μm using methods where the apertures are formed on the substrate as compared to methods where the apertures are pre-formed and then layers are assembled on a substrate. In embodiments, the first and second dielectric layers may be etchable layers, such as photo-imageable materials, and the method comprises etching, or photo-etching, to form the second aperture(s).

Additionally, in further embodiments of this method, the method comprises forming the first aperture(s) and/or well(s) in the first dielectric layer after the first dielectric layer has been provided to the substrate. This can enable accurate alignment of the first aperture/well with the electrode(s) to provide an improved and more reliable sensor response. In some embodiments, the step of forming the first aperture(s) and/or well(s) in the first dielectric layer may be prior to the provision of the second dielectric layer on the substrate or after the provision of the second dielectric layer but prior to forming the second aperture(s) in the second dielectric layer. In embodiments of the latter, the first aperture(s)/well(s) and second aperture(s) may be formed in the first dielectric layer and the second dielectric layer, respectively, after the first and second dielectric layers have been provided to the substrate. In one particular embodiment, the first aperture(s)/well(s) and second aperture(s) may be formed in the first dielectric layer and the second dielectric layer, respectively, after the first and second dielectric layers have been provided to the substrate, with the method comprising: forming the at least one second aperture in the second dielectric layer; and subsequently forming at least one first well/aperture in the first dielectric layer and within the at least one second aperture formed in the second dielectric layer. This ensures that the apertures are accurately aligned.

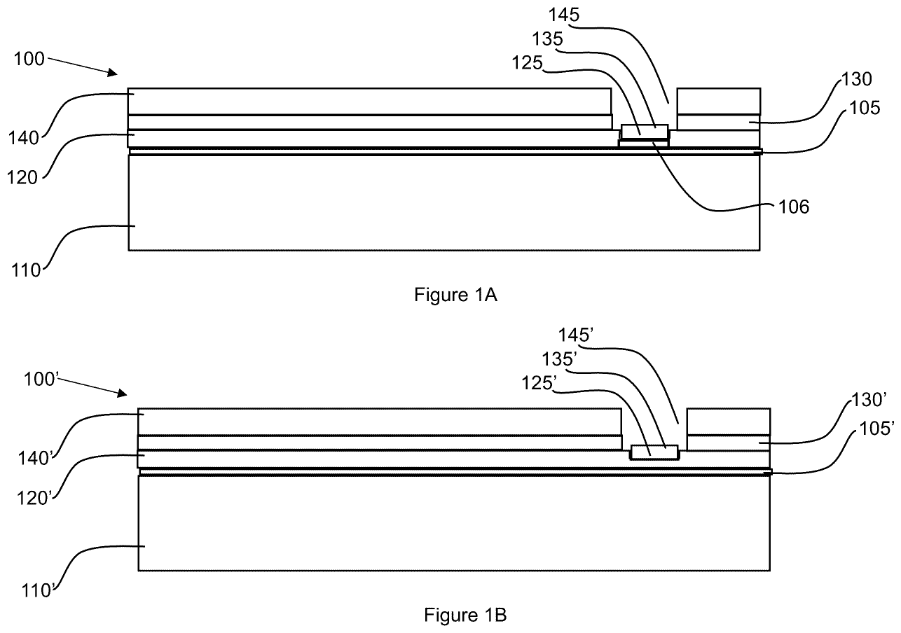

FIG. 1A shows a schematic cross-sectional view of a sensor assembly 100 for measuring a property of a sample. The sensor assembly 100 comprises a substrate 110 on which an electrode, a functional layer 106, a first dielectric layer 120, a second dielectric layer 130 and a stiffener layer 140 are formed.

Specifically, an electrode 105 is provided on the substrate 110 in the form of a metallic layer which extends across the width of the cross-section of the substrate 110. Although not depicted, the electrode 105 can be electrically connected to a signal processing unit for measuring a property of a sample provided to the sensor assembly 100.

On top of the electrode 105 is formed the first dielectric layer 120. The first dielectric layer 120 extends across the whole surface of the electrode 105 so as to electrically isolate the electrode 105 from an upper surface of the sensor assembly 100 with the exception of a first aperture 125 formed in the first dielectric layer 120 which extends from the upper surface of the first dielectric layer 120 to the lower surface of the first dielectric layer 120 and therefore exposes a portion of the electrode 105. In this embodiment, the functional layer 106, which in this case is a monolayer of capture species formed on the surface of electrode 105, is provided in this exposed region of the electrode 105. That is, the electrode 105 comprises the functional layer 106 across the region of the electrode 105 in fluid communication with the first aperture 125. In this way, a sample received within the first aperture 125 can interact with functional layer 106 and cause a change in the output from the electrode 105.

The second dielectric layer 130 is provided on the first dielectric layer 120. The second dielectric layer 130 comprises a second aperture 135 extending through the second dielectric layer 130 which is aligned with the first aperture 125 in the first dielectric layer 120 such that the upper surface of the second dielectric layer 130 is fluidly connected to the first aperture 125 in the first dielectric layer 120 and therefore the electrode 105 and functional layer 106. In this embodiment, the second aperture 135 has a largest diameter which is greater than the largest diameter of the first aperture 125, thereby providing a greater surface area for receipt of the sample fluid, thereby making sampling more straightforward. Moreover, this also contributes to the second aperture 135 having a larger volume than the first aperture 125, providing greater sampling ability but while ensuring that the first dielectric layer 120 can be used to provide the more complex structure adjacent the electrode 105 and incorporating the functional layer 106.

In this embodiment, the sensor assembly 100 further comprises a stiffener layer 140 on top of the second dielectric layer 130. The stiffener layer 140 is also provided with an aperture 145 which also aligns with the second aperture 135 in the second dielectric layer 130 and the first aperture 125 in the first dielectric layer 120. In this way, liquid sample contained within the apertures 125, 135, 145 is retained in contact with the functional layer 106 on the electrode 105. In embodiments where the substrate 110 is flexible, stiffener layers 140 are useful for providing mechanical support. That is, although the flexible nature of the substrate 110 is useful in some regions of the substrate 110, in those incorporating the first aperture 125 and second aperture 135, it may be advantageous to reinforce this with a stiffener layer 140. Exemplary stiffener layer 140 materials include polymers, such as polyimide.

FIG. 1B shows a schematic cross-sectional view of another embodiment of a sensor assembly 100′ for measuring a property of a sample. The structure of sensor assembly 100′ is identical to that of the sensor assembly 100 depicted in FIG. 1A, with the exception of the structure of the first dielectric layer 120′ and the absence of the functional layer 106.

The sensor assembly 100′ depicted in FIG. 1B therefore comprises a substrate 110′, an electrode 105′ provided on the substrate 110′; a first dielectric layer 120′ provided on the substrate 110′; and a second dielectric layer 130′ provided on the first dielectric layer 120′. In this embodiment, the sensor assembly 100′ further comprises a stiffener layer 140′ on top of the second dielectric layer 130′. These are arranged in the same manner as set out above for the embodiment of FIG. 1A.

The sensor assembly 100′ of this embodiment differs from that of the sensor assembly 100 of FIG. 1A in that the first dielectric layer 120′ comprises a well 125′ located therein rather than the first aperture 125 shown in FIG. 1A. Rather than providing a first aperture 125 which extends from the upper surface of the first dielectric layer 125 to the bottom surface of the first dielectric layer 120, the well 125′ has a base and thus is a recess with an opening in the upper surface of the first dielectric layer 120′ but does not extend the entire way therethrough. In this embodiment, the well 125′ therefore does not expose the electrode 105′ through the first dielectric layer 120′ but instead provides a region where the thickness of the first dielectric layer 120′ is thinner than the remainder of the first dielectric layer 120′. At this region, the electrode 105′ (located beneath the thinned portion in this embodiment) is responsive to the presence of a sample received within the well 125′. It will be appreciated that although the electrode 105′ and well 125′ in this embodiment are not functionalised, a functional layer could be provided on the inner surface of the well 125′, such as at the base or nadir of the well 125′.

The remaining structure is the same as that of FIG. 1A. For example, the second dielectric layer 130′ comprises a second aperture 135′ extending through the second dielectric layer 130′ which is aligned with the well 125′ such that the upper surface of the second dielectric layer 130′ is fluidly connected to the well 125′ in the first dielectric layer 120′.

In some embodiments, the depicted arrangement may be repeated several times over the substrate, such that there are plural wells 125′ (not shown). One of the plural wells 125′ may act as a control. In other embodiments, the well 125′ could be provided with pores so as to create a gas-sensing well.

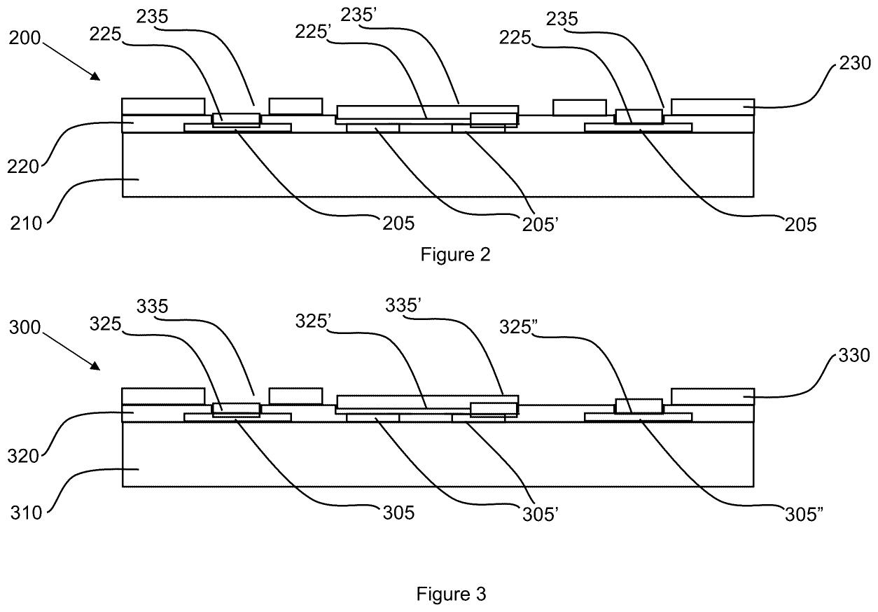

FIG. 2 shows a schematic cross-sectional view of a sensor assembly 200. As with the sensor assembly 100 of FIG. 1A, this sensor assembly 200 comprises a substrate 210, first and second pairs of electrodes 205, 205′ provided on the substrate 210; a first dielectric layer 220 provided on the substrate 210; and a second dielectric layer 230 provided on the first dielectric layer 220.

Specifically, plural electrodes 205, 205′ are provided on the substrate 210 in the form of discrete metallic layers. A first pair of electrodes 205 are provided on opposite sides of the substrate 210. A second pair of electrodes 205′ are provided in the centre of the substrate 210 between the electrodes of the first pair of electrodes 205. Although not depicted, the first and second pair of electrodes 205, 205′ can be electrically connected to a signal processing unit for measuring a property of a sample provided to the sensor assembly 200. In this embodiment, the first pair of electrodes 205 are functionalised with a first capture species so as to selectively bind to a first analyte. The second pair of electrodes 205′ are functionalised with a second (different) capture species and selectively bind to a second (different) analyte.

The first dielectric layer 220 is formed over the substrate 210 and is provided directly on the substrate 210 in the regions where the first and second pairs of electrodes 205, 205′ do not reside. The first dielectric layer 220 is accordingly provided between each of the plural electrodes in the first and second pairs of electrodes 205, 205; so as to electrically isolate the plural electrodes from one another. The first dielectric layer 220 is thicker than each of the plural electrodes of the first and second pairs of electrodes 205, 205′ so that, when provided on the substrate 210 during formation, it is formed over the first and second pairs of electrodes 205, 205′. However, the first dielectric layer 220 is also provided with plural apertures 225, 225′ which expose the first and second pairs of electrodes 205, 205′. In particular, there are two outer apertures 225 of the same size and a third, inner aperture 225′ which is larger than the two other apertures 225 and located between the two smaller apertures 225. The two smaller, outer apertures 225 are each associated with a single electrically isolated electrode of the first pair of electrodes 205 and the third, inner aperture 225′ exposes both of the two electrically isolated electrodes of the second pair of electrodes 205′. The first and second pair of electrodes 205, 205′ are accordingly embedded in the first dielectric layer 220 such that they are electrically isolated from one another but exposed through the two outer apertures 225 and the third, inner aperture 225′. As noted above, the second pair of electrodes 205′ located in the third, inner aperture 225′ are commonly functionalised but electrically isolated by the first dielectric layer 220.

The second dielectric layer 230 is provided on the first dielectric layer 220 and comprises three apertures 235, 235′ extending through the second dielectric layer 230, each of which is aligned with a corresponding aperture 225, 225′ in the first dielectric layer 220 such that the upper surface of the second dielectric layer 230 is fluidly connected to a respective aperture 225, 225′ in the first dielectric layer 220. Specifically, the second dielectric layer 230 is provided with two outer apertures 235 of the same size which correspond to and are in fluid communication with the two outer apertures 225 of the first dielectric layer 220. The second dielectric layer 230 also comprises a third, inner aperture 235′ which corresponds to and is in fluid communication with the third, inner aperture 225′ of the first dielectric layer 220. Each of the apertures 235, 235′ in the second dielectric layer 230 is larger in diameter and volume than the respective aperture 225, 225′ in the first dielectric layer 220.

Adhesive is provided between the first dielectric layer 220 and the second dielectric layer 230.

FIG. 3 shows a schematic cross-sectional view of a sensor assembly 300. As with the sensor assembly 300 of FIG. 2, this sensor assembly 300 comprises a substrate 310, first and second pairs of electrodes 305, 305′ provided on the substrate 310; a first dielectric layer 320 provided on the substrate 310; and a second dielectric layer 330 provided on the first dielectric layer 320.

In this embodiment, the configuration of the electrodes 305, 305′ and the first dielectric layer 320 is identical to the corresponding parts of the sensor assembly 200 of FIG. 2. In this embodiment, the difference arises in the configuration of the second dielectric layer 330. As such, the sensor assembly 300 of this embodiment comprises a first pair of electrodes 305, 305″ comprised of a first electrode 305 provided on one side of the substrate 310 and a second electrode 205″ provided on the opposite side of the substrate 310. A second pair of electrodes 305′ are provided in the centre of the substrate 310 between the electrodes of the first pair of electrodes 305. The first dielectric layer 320 is formed over the substrate 310 and the first and second pairs of electrodes 305, 305′, 305″. The first dielectric layer 320 is also provided with plural apertures 325, 325′ which expose the first and second pairs of electrodes 305, 305′, 305″. In particular, there are two outer apertures 325, 325″ of the same size and a third, inner aperture 325′ which is larger than the two outer apertures 325, 325″ and located between the two smaller outer apertures 325, 325″. The two smaller, outer apertures 325, 325″ are each associated with a single electrically isolated electrode of the first pair of electrodes 305, 305″ and the third, inner aperture 325′ exposes both of the two electrically isolated electrodes of the second pair of electrodes 305′. The first and second pair of electrodes 305, 305′, 305″ are accordingly embedded in the first dielectric layer 320 such that they are electrically isolated from one another but exposed through the two outer apertures 325, 325″ and the third, inner aperture 325′.

The second dielectric layer 330 is provided on the first dielectric layer 320 and comprises two upper apertures 335, 335′ (upper denoting that they are part of the second dielectric layer 330) extending through the second dielectric layer 330. These are arranged such that the first upper aperture 335 is aligned with a first one of the two outer apertures 325 in the first dielectric layer 320 and the second, upper aperture 335′ in the second dielectric layer 330 is aligned with (i.e. encompasses and provides fluid communication with) the other outer aperture 325″ in the first dielectric layer 320 and also the two inner apertures 325′ in the first dielectric layer 320.

Accordingly, each of the apertures 325, 325′, 325″ in the first dielectric layer 320 is associated with one electrode from the first and second pairs of electrodes 305, 305′, 305″.

Accordingly, this arrangement ensures that the sample contacting the second dielectric layer 330 can be provided to two different electrode arrangements, if required, and the second upper aperture 335′ provide a large volume aperture which can distribute sample to three distinct electrodes. This provides versatility in the use of the sensor assembly 300.

FIG. 4 depicts a schematic plan view of a sensor assembly 400 in another embodiment. The sensor assembly 400 comprises a substrate (not visible), plural sets of electrodes 405, 405′, 405″; a first dielectric layer 420 provided on the substrate; and a second dielectric layer 430 provided on the first dielectric layer 420.

In this embodiment, the sensor assembly 400 is arranged as a circular structure to correspond with the shape of a droplet of liquid provided on top of the sensor assembly 400 and defines a central axis. Three sets of electrodes 405, 405′, 405″ are provided arranged as concentric circles around the central axis of the circular structure.

The first set of electrodes comprises a single electrode 405 located on the central axis in the centre of sensor assembly 400. It is exposed to the upper surface of the sensor assembly 400 through a first aperture 425 in the first dielectric layer 420 and a second aperture 435 in the second dielectric layer 430. The second set of electrodes comprises 16 separate electrodes 405′ arranged in a concentric circle about the central electrode 405′. Each of the electrodes 405′ is exposed to the upper surface of the sensor assembly 400 by a corresponding first aperture 425′ in the first dielectric layer 420, such that there are 16 separate apertures 425′ the first dielectric layer 420 in this concentric circle, and a corresponding second aperture 435′ in the second dielectric layer 430. For this set of electrodes 405, there are eight second apertures 435′ in the second dielectric layer 430 with each of the second apertures 435′ connected to two first apertures 425′ in the first dielectric layer 420, and thus each of the second apertures 435′ in this concentric circle exposes two electrodes 405′. The electrodes 405′ of the second set of electrodes are each round in this embodiment (but other shapes are envisaged). The third set of electrodes comprises four electrodes 405″ each having an arc shape and arranged around the central axis in a radially symmetric manner to form an outermost concentric circle. Each of the electrodes 405″ is exposed through a first aperture 425″ through the first dielectric layer 420 and a corresponding second aperture 435″ through the second dielectric layer 430. Accordingly, the three sets of electrodes 405, 405′, 405″, first apertures 425, 425′, 425″ in the first dielectric layer 420 and second apertures 435, 435′, 435″ in the second dielectric layer 430 are arranged so as to be radially symmetric. Moreover, each of the second apertures 435, 435′, 435″ in the second dielectric layer 430 has a larger volume than the corresponding first apertures 425, 425′, 425″ in the first dielectric layer 420 (i.e., those first apertures 425, 425′, 425″ in the first dielectric layer 420 to which the second apertures 435, 435′, 435″ are fluidly connected).

In this embodiment, each of the first, second and third set of electrodes 405, 405′, 405″ is arranged to detect a different property. For example, the electrodes 405″ of the third set of electrode may be configured to sense a property such as conductivity, which could be used to confirm that a droplet of fluid has been received on the upper surface of the sensor assembly 400 and has spread to the edges of the sensor assembly 400 so that it can be assumed that it is in contact with the first and second sets of electrodes 405, 405′. The second set of electrodes 405′ and central electrode 405 of the first set may each be functionalised (e.g., by a capture species adhered to the surface of the electrodes 405, 405′). Alternatively, the central electrode 405 of the first set could be a reference or counter electrode. Each of the first, second and third sets of electrodes therefore can provide a different sensing function and defines different functional regions.

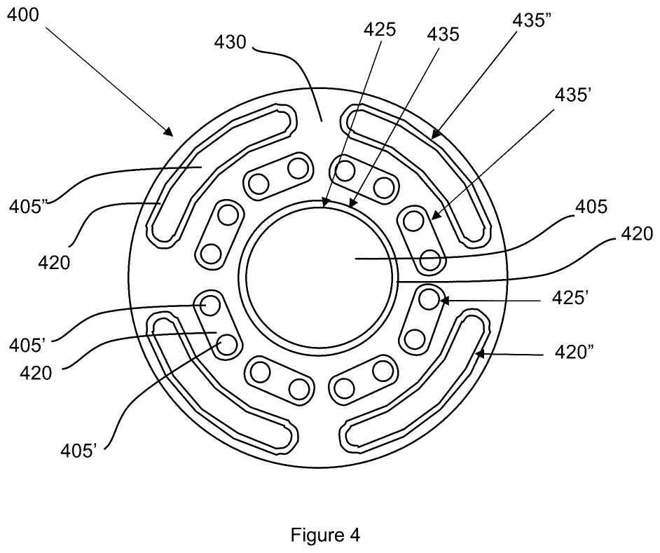

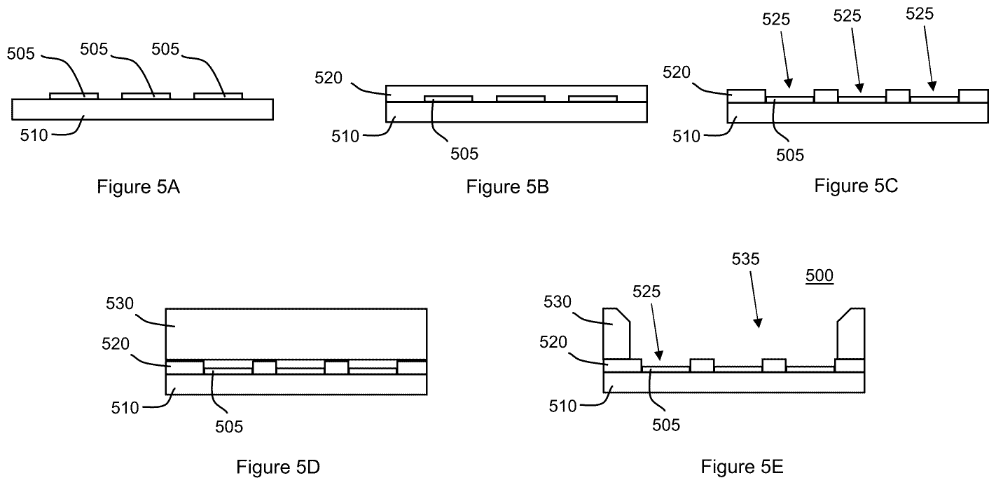

FIGS. 5A to 5E schematically depict the manufacture of a sensor assembly 500 according to an embodiment.

The method first comprises providing a substrate 510 and forming at least one electrode 505 on the substrate 510, as depicted in FIG. 5A. In this embodiment, three electrodes 505 are provided as three distinct metallic layers on the upper surface of the substrate 510.

The method further comprises providing a first dielectric layer 520 on the substrate 510, as depicted in FIG. 5B. In this embodiment, the first dielectric layer 520 is a photo-imageable polymeric layer formed over the substrate 510 and the three electrodes 505. The first dielectric layer 520 is provided between the three electrodes 505 so that these are electrically isolated by the first dielectric layer 520 and, in this initial step, are fully covered by the first dielectric layer 520.

In this embodiment, the method further comprises forming three first apertures 525 in the first dielectric layer 520, as depicted in FIG. 5C. In this embodiment, this is prior to the provision of any subsequent layers but after the first dielectric layer 520 is provided on the substrate. The three first apertures are each formed over one of the three electrodes 505 and have the same shape and size (from a plan view) as the electrodes 505. In this way, the upper surface of each of the three electrodes 505 is exposed through the first dielectric layer 520. The electrodes 505 can therefore be responsive to the presence of a sample received within the first apertures 525. It will be appreciated that the formation of the first apertures 525 can be using any means used in the art, for example using an etching process or drilling process (such as laser drilling) on the layer formed on the substrate. One advantageous method includes photo-imaging the photo-imageable polymer used to form the first dielectric layer 520.

The method further comprises providing a second dielectric layer 530 on the first dielectric layer 520, as depicted in FIG. 5D. This covers the three first apertures 525 in the first dielectric layer 520.

The method further comprises forming a second aperture 535 through the second dielectric layer 530, as depicted in FIG. 5E. In this embodiment a single second aperture 535 is formed which extends over all three of the first apertures 525 and the corresponding electrodes 505. Each of the three electrodes 505 and first apertures 525 is accordingly fluidly connected to the second aperture 535 in the second dielectric layer. This second aperture 535 may be formed using the techniques discussed in respect of the first apertures 525. Forming the first and second apertures 525, 535 in this way can advantageously avoid the difficulties associated with aligning pre-formed apertures.

The second dielectric layer 530 in this embodiment is significantly thicker than the first dielectric layer 520 providing the second aperture 535 with a substantial capacity for liquid sample. This allows for a user to easily provide the sample to the sensor assembly 500 (e.g., spot), but subsequently the fluid can be distributing via the first apertures 525 to the electrodes 505, which are all on a much smaller scale. This ensures that adequate sample is provided to the electrodes 505, as compared to if a user was to attempt to provide sample to each of the electrodes 505 without the second aperture 535.

Although not depicted, in some further embodiments of the method set out in FIGS. 5A to 5E, the electrodes 505 could be functionalised. This could be prior to the application of the first dielectric layer 520, for example, or could be after the application of the first and/or second dielectric layers 520, 530. This may depend on the nature of the functionalisation, since capture species can be prone to damage during processing. The method set out herein advantageously allows for formation at the end of the manufacturing process since the first apertures can be used to create the necessary structures to retain the capture species during functionalisation or on which the capture species can be formed.

It should be understood that the detailed description and specific examples, while indicating exemplary embodiments of the apparatus, systems and methods, are intended for purposes of illustration only and are not intended to limit the scope. These and other features, aspects, and advantages of the apparatus, systems and methods of the present invention can be better understood from the description, appended claims or aspects, and accompanying drawings. It should be understood that the Figures are merely schematic and are not drawn to scale. It should also be understood that the same reference numerals are used throughout the figures to indicate the same or similar parts.

Other variations to the disclosed embodiments can be understood and effected by those skilled in the art in practicing the disclosure, from a study of the drawings, the disclosure, and the appended aspects or claims. In the aspects or claims, the word “comprising” does not exclude other elements or steps, and the indefinite article “a” or “an” does not exclude a plurality. The mere fact that certain measures are recited in mutually different dependent aspects or claims does not indicate that a combination of these measures cannot be used to advantage. Any reference signs in the claims should not be construed as limiting the scope.

1. A sensor assembly for measuring a property of a sample, comprising:
a substrate;
at least one electrode provided on the substrate;
a first dielectric layer provided on the substrate, the first dielectric layer comprising a well or aperture associated with the electrode such that the electrode is responsive to the presence of a sample received within the well or aperture; and
a second dielectric layer provided on the first dielectric layer comprising an aperture extending through the second dielectric layer fluidly connected to the well or aperture in the first dielectric layer.

2. The sensor assembly of claim 1, wherein the at least one electrode is functionalised so as to interact with a sample received within the well or aperture of the first dielectric layer.

3. The sensor assembly of claim 2, wherein the at least one electrode comprises a capture species configured to selectively interact with an analyte within a sample.

4. The sensor assembly of claim 1, wherein the sensor assembly comprises a set of electrodes comprising plurality of electrodes, and wherein the first dielectric layer comprises a plurality of wells and/or apertures, with at least one well or aperture associated with each electrode of the set of electrodes.

5. The sensor assembly of claim 4, wherein second dielectric layer comprises a plurality of apertures extending through the second dielectric layer, each aperture corresponding to and being fluidly connected to one of the plurality of wells or apertures in the first dielectric layer.

6. The sensor assembly of claim 4, wherein the plurality of electrodes of the set of electrodes are separated from one another by the first dielectric layer.

7. The sensor assembly of claim 4, wherein the sensor assembly comprises: a first functionalised region comprising at least one of the wells or apertures in the first dielectric layer, at least one electrode and a first capture species configured to selectively bind to a first analyte and provided on at least a part of the first functionalised region; and a second functionalised region comprising at least one of the wells or apertures in the first dielectric layer, at least one electrode and a second capture species configured to selectively bind to a second analyte and provided on at least a part of the second functionalised region.

8. The sensor assembly of claim 1, wherein a volume of the aperture or each aperture of the second dielectric layer is greater than a volume of the well or aperture or each well or aperture of the first dielectric layer.

9. The sensor assembly of claim 1, wherein the first dielectric layer and/or the second dielectric layer comprises a polymer.

10. The sensor assembly of claim 9, wherein the first dielectric layer and/or the second dielectric layer comprises polyimide, polyethylene terephthalate or a combination thereof.

11. The sensor assembly of claim 1, wherein the first dielectric layer has a thickness of from 1 μm to 50 μm; and/or, wherein the second dielectric layer has a thickness of from 50 μm to 1000 μm.

12. The sensor assembly of claim 1, wherein the substrate is a flexible substrate.

13. The sensor assembly of claim 1, wherein the electrode comprises a plurality of metal layers, the plurality of metal layers comprising or being formed from copper, nickel, platinum, silver, silver chloride, gold, or other noble metals.

14. A method of forming a sensor assembly, the method comprising:
providing a substrate;
forming at least one electrode on the substrate;
providing a first dielectric layer on the substrate; and
providing a second dielectric layer on the first dielectric layer,
wherein the first dielectric layer comprises a well or aperture associated with the electrode such that the electrode is responsive to the presence of a sample received within the well or aperture; and
wherein the second dielectric layer comprises an aperture extending through the second dielectric layer and is fluidly connected to the well or aperture in the first dielectric layer.

15. The method of claim 14, wherein the method further comprises forming a well or aperture in the first dielectric layer, the well or aperture being associated with the electrode such that the electrode is responsive to the presence of a sample received within the well or aperture.

16. The method of claim 15, wherein forming a well or aperture in the first dielectric layer is carried out before providing the second dielectric layer on the first dielectric layer.

17. The method of claim 14, wherein the method further comprises forming an aperture in the second dielectric layer, the aperture extending through the second dielectric layer to fluidly connect with the well or aperture in the first dielectric layer.

18. The method of claim 17, wherein forming the aperture in the second dielectric layer is carried out after providing the second dielectric layer on the first dielectric layer.

19. The method of claim 18, wherein forming the well or aperture in the first dielectric layer is carried out after the first dielectric layer has been provided to the substrate.

20. The method of claim 19, wherein forming the well or aperture in the first dielectric layer is carried out prior to providing the second dielectric layer on the substrate.
